(19) United States
(12) Patent Application Publication
(10) Pub. No.: US 2026/0071257 A1
(43) Pub. Date: Mar. 12, 2026

(54) DETECTION PLATFORM FOR UNLABELED OLIGONUCLEOTIDES

(71) Applicant: Georgia Tech Research Corporation, Atlanta, GA (US)

(72) Inventors: Valeria Tohver Milam, Atlanta, GA (US); Mary Catherine Adams, Atlanta, GA (US)

(21) Appl. No.: 18/707,940

(22) PCT Filed: Nov. 8, 2022
(86) PCT No.: PCT/US2022/079434

§ 371 Date: May 7, 2024

Related U.S. Application Data
(60) Provisional application No. 63/277,017, filed on Nov. 8, 2021.

(51) Int. Cl. C12Q 1/6818; C12Q 1/6809; C12Q 1/6876; C12Q 1/70

(52) U.S. Cl. CPC C12Q 1/6818; C12Q 1/6809; C12Q 1/6876; C12Q 1/701

(57) ABSTRACT

A composition comprising microspheres and at least one double-stranded nucleic acid (dsNA) molecule. One strand of the at least one dsNA molecule is bound to an outer surface of the microspheres at a 3′_end. The bound strand of the at least one dsNA molecule comprises a recognition domain and contains a quenchable colorimetric indicator at a 5′ end. The other strand of the at least one dsNA molecule is soluble and contains a quencher of the colorimetric indicator at a 3′ end. The present disclosure also provides methods of making a composition comprising microspheres and at least one double-stranded nucleic acid (dsNA) molecule.

### CROSS-REFERENCE TO RELATED APPLICATIONS

This application claims the benefit of U.S. Provisional Application Ser. No. 63/277,017, filed on 8 Nov. 2021, which is incorporated herein by reference in its entirety as if fully set forth below.

### FEDERALLY SPONSORED RESEARCH STATEMENT

This invention was made with government support under grant/award number 1829137 awarded by the National Science Foundation, and grant/award number P200A180076, awarded by the US Department of Education Polymeric Materials Science and Engineering (PMSE) GAANN Fellowship. The government has certain rights in the invention.

### FIELD OF THE DISCLOSURE

The various embodiments of the present disclosure relate generally to systems and methods for making and using nucleic acid detection platforms, and more particularly to nucleic acid detection platforms free from expensive PCR instrumentation or methods requiring multi-step manufacturing steps.

### BACKGROUND

Reliable, practical diagnostic platforms present an enabling pathway to minimize the spread of pathogens by quickly identifying an individual carrier who can then be isolated. Detection platforms often require expensive and specialized equipment and/or a variety of reagents combined with a number of preparatory steps. During supply shortages, these limitations inhibit universal use.

Detection platforms that use more readily available equipment and reagents such as molecular beacons (i.e., self-folded hairpins) and double-stranded nucleic acid probes (dsprobes) typically involve fluorescence spectroscopy of oligonucleotide solutions initially in a quenched or signal-off state. Among the challenges for molecular beacons is their susceptibility to unfolding in the absence of target as the self-hybridized stem segment undergoes spontaneous “fraying and peeling” into an unhybridized sequence that may result in false positives. Further, detecting the presence of relatively short (e.g. 10-30 bases in length) oligonucleotide sequences has been challenging as the detection of short oligonucleotide sequences relies on promoting hybridization activity of the targeted sequence with a matching probe sequence. There is a need for a high throughput and specific detection platform that can easily detect the presence of a pathogen without expensive machinery, high demand medical equipment, and multiple processing steps due to fluorescently-labeled targets.

### BRIEF SUMMARY

The present disclosure relates to systems and methods for making and using nucleic acid detection platforms. An exemplary embodiment of the present disclosure provides a composition comprising microspheres and at least one double-stranded nucleic acid (dsNA) molecule. One strand of the at least one dsNA molecule can be bound to an outer surface of the microspheres at a 3′end. The bound strand of the at least one dsNA molecule can include a recognition domain. The bound strand can also contain a quenchable colorimetric indicator at a 5′ end. The other strand of the at least one dsNA molecule can be soluble and can contain a quencher of the colorimetric indicator at a 3′ end.

In any of the embodiments disclosed herein, the microspheres can be coated in streptavidin and the bound strand of the at least one dsNA molecule comprises biotin at 3′ end.

In any of the embodiments disclosed herein, the bound strand of the at least one dsNA molecule further can include a toehold domain at 3′ end. The toehold domain can include between 1 and 12 nucleotides.

In any of the embodiments disclosed herein, there can be at least one mismatched base pair between the bound strand of the at least one dsNA molecule and the soluble strand of the at least one dsNA molecule in either or both of the recognition domain and the toehold domain.

In any of the embodiments disclosed herein, the quenchable colorimetric indicator can be a fluorescent indicator.

In any of the embodiments disclosed herein, the soluble strand of the at least one dsNA molecule can be configured to be displaced by a single stranded NA molecule, thereby displacing the quencher of the colorimetric indicator such that the colorimetric indicator is active. The active colorimetric indicator can be detectable and/or quantifiable.

In any of the embodiments disclosed herein, the dsNA molecule can include DNA, RNA, or LNA.

In any of the embodiments disclosed herein, the bound strand of the at least one dsNA molecule can include a COVID-19-specific nucleic acid, or encodes a COVID-19-specific protein.

In any of the embodiments disclosed herein, the bound strand of the at least one dsNA molecule can have a sequence selected from the group consisting of SEQ ID NOs: 1, 12, and 21. The soluble strand of the at least one dsNA molecule can have a sequence selected from the group consisting of SEQ ID NOs: 2-11, 13-20, and 22.

An exemplary embodiment of the present disclosure provides a kit for detecting the presence of a nucleic acid in a sample. The kit can include microspheres and at least one double-stranded nucleic acid (dsNA) molecule. One strand of the at least one dsNA molecule can be bound to an outer surface of the microspheres at a 3′end. The bound strand of the at least one dsNA molecule can include a recognition domain. The bound strand can also contain a quenchable colorimetric indicator at a 5′ end. The other strand of the at least one dsNA molecule can be soluble and can contain a quencher of the colorimetric indicator at a 3′ end.

In any of the embodiments disclosed herein, the microspheres can be coated in streptavidin and the bound strand of the at least one dsNA molecule comprises biotin at 3′ end.

In any of the embodiments disclosed herein, the bound strand of the at least one dsNA molecule further can include a toehold domain at 3′ end. The toehold domain can include between 1 and 12 nucleotides.

In any of the embodiments disclosed herein, there can be at least one mismatched base pair between the bound strand of the at least one dsNA molecule and the soluble strand of the at least one dsNA molecule in either or both of the recognition domain and the toehold domain.

In any of the embodiments disclosed herein, the quenchable colorimetric indicator can be a fluorescent indicator.

In any of the embodiments disclosed herein, the soluble strand of the at least one dsNA molecule can be configured to be displaced by a single stranded NA molecule, thereby displacing the quencher of the colorimetric indicator such that the colorimetric indicator is active.

In any of the embodiments disclosed herein, the active colorimetric indicator can be detectable and/or quantifiable.

In any of the embodiments disclosed herein, the dsNA molecule can include DNA, RNA, or LNA.

In any of the embodiments disclosed herein, the bound strand of the at least one dsNA molecule can include a COVID-19-specific nucleic acid, or encodes a COVID-19-specific protein.

In any of the embodiments disclosed herein, the bound strand of the at least one dsNA molecule can include a sequence selected from the group consisting of SEQ ID NOs: 1, 12, and 21. The soluble strand of the at least one dsNA molecule can have a sequence selected from the group consisting of SEQ ID NOs: 2-11, 13-20, and 22.

In any of the embodiments disclosed herein, the sample can include a biological sample, a water sample, a wastewater sample, an environmental sample, a food sample, and/or a food contact surface sample.

In any of the embodiments disclosed herein, the biological sample can include tissue, blood, serum, plasma, saliva, nasopharyngeal swab or secretion, urine, cerebrospinal fluid, lymphatic fluid, sputum, cell lysate, and the like.

An exemplary embodiment of the present disclosure provides a method of detecting and/or quantifying a nucleic acid. The method can include incubating the nucleic acid with a composition and measuring the colorimetric indicator to detect and/or quantify the nucleic acid. The composition can include microspheres and at least one double-stranded nucleic acid (dsNA) molecule. One strand of the at least one dsNA molecule can be bound to an outer surface of the microspheres at a 3′end. The bound strand of the at least one dsNA molecule can include a recognition domain. The bound strand can also contain a quenchable colorimetric indicator at a 5′ end. The other strand of the at least one dsNA molecule can be soluble and can contain a quencher of the colorimetric indicator at a 3′ end.

In any of the embodiments disclosed herein, the measuring step can be performed by a high-throughput spectrophotometer, a fluorescence activated cell sorting (FACS) system, or a color activated cell sorting system.

In any of the embodiments disclosed herein, the microspheres can be coated in streptavidin and the bound strand of the at least one dsNA molecule comprises biotin at 3′ end.

In any of the embodiments disclosed herein, the bound strand of the at least one dsNA molecule further can include a toehold domain at 3′ end.

In any of the embodiments disclosed herein, the toehold domain can include between 1 and 12 nucleotides.

In any of the embodiments disclosed herein, there can be at least one mismatched base pair between the bound strand of the at least one dsNA molecule and the soluble strand of the at least one dsNA molecule in either or both of the recognition domain and the toehold domain.

In any of the embodiments disclosed herein, the quenchable colorimetric indicator can be a fluorescent indicator.

In any of the embodiments disclosed herein, the dsNA molecule can include DNA, RNA, or LNA.

In any of the embodiments disclosed herein, the bound strand of the at least one dsNA molecule can include a COVID-19-specific nucleic acid, or encodes a COVID-19-specific protein.

In any of the embodiments disclosed herein, the bound strand of the at least one dsNA molecule can have a sequence selected from the group consisting of SEQ ID NOs: 1, 12, and 21. The soluble strand of the at least one dsNA molecule can have a sequence selected from the group consisting of SEQ ID NOs: 2-11, 13-20, and 22.

An exemplary embodiment of the present disclosure provides a method of detecting a nucleic acid in a biological sample from a patient. The method can include isolating the nucleic acid from the biological sample, incubating the nucleic acid with a composition, and measuring the colorimetric indicator to detect and/or quantify the nucleic acid. The composition can include microspheres and at least one double-stranded nucleic acid (dsNA) molecule. One strand of the at least one dsNA molecule can be bound to an outer surface of the microspheres at a 3′end. The bound strand of the at least one dsNA molecule can include a recognition domain. The bound strand can also contain a quenchable colorimetric indicator at a 5′ end. The other strand of the at least one dsNA molecule can be soluble and can contain a quencher of the colorimetric indicator at a 3′ end.

In any of the embodiments disclosed herein, the measuring step can be performed by a high-throughput spectrophotometer, a fluorescence activated cell sorting (FACS) system, or a color activated cell sorting system.

In any of the embodiments disclosed herein, the microspheres can be coated in streptavidin and the bound strand of the at least one dsNA molecule comprises biotin at 3′ end.

In any of the embodiments disclosed herein, the bound strand of the at least one dsNA molecule further can include a toehold domain at 3′ end.

In any of the embodiments disclosed herein, the toehold domain can include between 1 and 12 nucleotides.

In any of the embodiments disclosed herein, there can be at least one mismatched base pair between the bound strand of the at least one dsNA molecule and the soluble strand of the at least one dsNA molecule in either or both of the recognition domain and the toehold domain.

In any of the embodiments disclosed herein, the quenchable colorimetric indicator can be a fluorescent indicator.

In any of the embodiments disclosed herein, the dsNA molecule can include DNA, RNA, or LNA.

In any of the embodiments disclosed herein, the bound strand of the at least one dsNA molecule can include a COVID-19-specific nucleic acid, or encodes a COVID-19-specific protein.

In any of the embodiments disclosed herein, the bound strand of the at least one dsNA molecule can have a sequence selected from the group consisting of SEQ ID NOs: 1, 12, and 21. The soluble strand of the at least one dsNA molecule can have a sequence selected from the group consisting of SEQ ID NOs: 2-11, 13-20, and 22.

In any of the embodiments disclosed herein, the patient can be a mammal. The patient can be a human or veterinary animal.

In any of the embodiments disclosed herein, the biological sample can include tissue, blood, serum, plasma, saliva, nasopharyngeal swab or secretion, urine, cerebrospinal fluid, lymphatic fluid, sputum, cell lysate, and the like.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a schematic of an example of a responsive nucleic acid detection platform, in accordance with an exemplary embodiment of the present invention.

FIG. 2 provides a schematic of an example of an unresponsive nucleic acid detection platform, in accordance with an exemplary embodiment of the present invention.

FIG. 3 provides a schematic of an example nucleic acid detection platform with initial fluorescent signals, in accordance with an exemplary embodiment of the present invention.

FIG. 4 provides a bar graph of molecules of equivalent soluble fluorochrome (MESF) for example nucleic acid detection platforms with addition of various sequences, in accordance with an exemplary embodiment of the present invention.

FIG. 5 provides a bar graph of molecules of equivalent soluble fluorochrome (MESF) for example nucleic acid detection platforms with addition of various sequences, in accordance with an exemplary embodiment of the present invention.

FIG. 6 provides a bar graph of molecules of equivalent soluble fluorochrome (MESF) for an example nucleic acid detection platform with addition of various sequences, in accordance with an exemplary embodiment of the present invention.

FIG. 7 provides a flow-chart of an example method of making nucleic acid detection platforms, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

By “comprising” or “containing” or “including” is meant that at least the named compound, element, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another. It is also noted that terms like “substantially” and “about” are utilized herein to represent the inherent degree of uncertainty that may be attributed to any quantitative comparison, value, measurement, or other representation.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

In general, nucleic acid detection platforms involving double-stranded (ds) probes comprised of quencher-dye sequence pairs exhibit advantages over single-stranded probes including superior target sequence specificity and no prerequisite target labeling. Further, dsprobe design can include finding optimal sequence combinations that balance stability requirements while minimizing spontaneous dsprobe dissociation events (i.e., maintain signal-off state in target absence) with fast, accurate response to a specific target sequence (i.e., selectively trigger signal-on state in target presence).

In some embodiments, flow cytometry can be used to rapidly interrogate the stability and selective responsiveness of locked nucleic acid (LNA; i.e., a nucleic acid comprising one or more locked nucleotides), RNA dsprobes, and DNA dsprobes to a segment of a pathogen. For instance, the 24 base-long segment of SARS-COV-2 RNA and approximately 243 similar RNA sequences (acting as example SARS-COV-2 variants) can be detected using the system described herein. In a preferred embodiment, a DNA dsprobe with a 15 base-long hybridization partner containing a central abasic site can exhibit very stable, and selective detection of a pathogen such as SARS-COV-2 RNA.

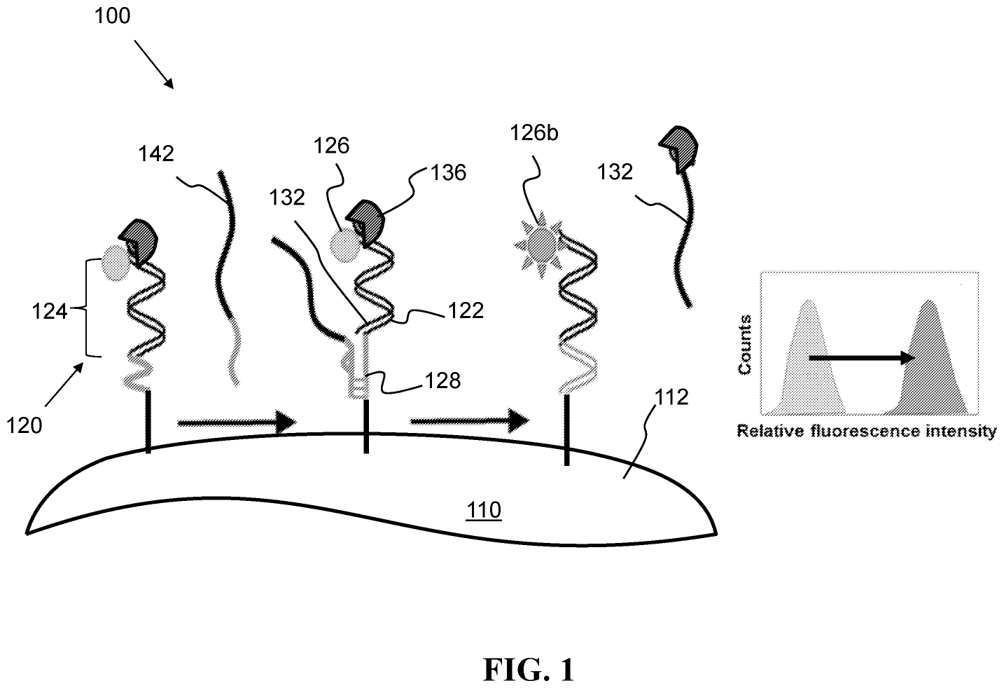

As shown in FIG. 1, an exemplary embodiment of the present invention provides an example composition 100 of a nucleic acid detection platform in an initially signal-off state. Composition 100 can undergo displacement of one of the sequences by a target sequence 142 to induce a signal from the platform, as indicated in a shift in fluorescence intensity in the plot to the right. Composition 100 can include microspheres 110 or nanobeads functionalized with double-stranded nucleic acid (dsNA) molecules 120.

In some embodiments, the dsNA molecule can be dsRNA or dsDNA. In some embodiments, the nucleic acid molecules can include one or more locked nucleotides to form a locked nucleic acid (“LNA”). In a preferred embodiment, a dsNA probe can include a ratio of approximately 33% LNA to 67% DNA, although lower amounts of LNA are possible (e.g., 32% LNA to 68% DNA; 30% LNA to 70% DNA; 25% LNA to 75% DNA; 20% LNA to 80% DNA; 15% LNA to 85% DNA; 10% LNA to 90% DNA; 5% LNA to 95% DNA, and any ratio in between, e.g., 28.4% LNA to 71.6% DNA).

Microparticles, or microspheres 110 can be nonfluorescent or fluorescently-labeled and useful in flow cytometry, confocal laser scanning microscopy, light scattering measurements, or particle dynamic analysis. Microspheres 110 can be made with polystyrene or melamine resin, modified with surface functional groups (e.g., methylol groups, amino groups, carboxylate groups, streptavidin, and the like). As would be appreciated by one of skill in the art, microspheres can be prepared with variations in chemical composition, size, type of fluorochrome (optional), and surface functional groups. Microparticles can range from about 0.1 μm to about 100 μm (e.g., from about 0.1 μm to about 1 μm, from about 1 μm to about 3 μm, from about 2 μm to about 4 μm, from about 3 μm to about 5 μm, from about 4 μm to about 6 μm, from about 5 μm to about 7 μm, from about 6 μm to about 8 μm, from about 7 μm to about 9 μm, from about 8 μm to about 10 μm, from about 10 μm to about 30 μm, from about 20 μm to about 40 μm, from about 30 μm to about 50 μm, from about 40 μm to about 60 μm, from about 50 μm to about 70 μm, from about 60 μm to about 80 μm, from about 70 μm to about 90 μm, from about 80 μm to about 100 μm, and any size in between, e.g., from about 0.86 μm to about 37 μm). Suitable fluorochromes can include FITC, green fluorescence, rhodamine B, orange fluorescence, nile blue A, red fluorescence, and the like. In some embodiments, microspheres 110 can be used to provide consistent instrument performance with minimal data variation for alignment, size calibration, and sorting set-up; convenience to save sample and set compensation for anti-body, reagent, and fluorescent proteins or nucleic acids; and comparable data between samples and instruments by acting as a standard.

In some embodiments, composition 100 may include nanobeads that can offer intense and stable fluorescent signals. For instance, polyacrylnitrile, polystyrene, or PD nanoparticles can be fluorescently labeled and can be less than about 50 nm in diameter (e.g., less than 45 nm, less than a 40 nm, less than 35 nm, less than a 30 nm, less than 25 nm, less than a 20 nm, less than 15 nm, less than a 10 nm, less than 5 nm, and any size in between, e.g., less than about 16.2 nm).

Referring back to FIG. 1, microsphere 110 can have a surface 112 functionalized such that one strand 122 of the dsNA molecule 120 can be bound at a 3′ end of strand 122. The dsNA molecule can include DNA, RNA, or locked nucleic acid (LNA).

In some embodiments, microsphere surface 112 is functionalized with streptavidin to form a strong, but noncovalent bond with strand 122 with a biotin at 3′ end. Bound strand 122 can further include a recognition domain 124 extending along at least a portion of the bound strand 122 between 3′ and 5′ ends. In some examples, recognition domain 124 can make up approximately 20% to approximately 99% of the sequence of bound strand 122 (e.g., approximately 22%, approximately 24%, approximately 26%, approximately 28%, approximately 30%, approximately 32%, approximately 34%, approximately 36%, approximately 38%, approximately 40%, approximately 42%, approximately 44%, approximately 46%, approximately 48%, approximately 50%, approximately 52%, approximately 54%, approximately 56%, approximately 58%, approximately 60%, approximately 62%, approximately 64%, approximately 66%, approximately 68%, approximately 70%, approximately 72%, approximately 74%, approximately 76%, approximately 78%, approximately 80%, approximately 82%, approximately 84%, approximately 86%, approximately 88%, approximately 90%, approximately 92%, approximately 94%, approximately 96%, approximately 98%, approximately 99%, or any range in between, e.g., approximately 97.5%). In a preferred embodiment, the recognition domain comprises at least 50% nucleic acids in the recognition segment.

Bound strand 122 can also include a toehold domain 128. Toehold domain 128 can be positioned nearer 3′ end of all bound strands such that a target may hybridize with the bound strands in a similar way. In some embodiments, toehold domain 128 can be positioned near 3′ end of one bound strand, and further from 3′ end of a neighboring bound strand such that a target may hybridize with the bound strand in a staggered manner along microsphere. In some embodiments, the toehold domain 128 may include from about 1 nucleotide to about 12 nucleotides (e.g., 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides). In a preferred embodiment, the toehold domain comprises at least 6 nucleotides.

Bound strand 122 may also include a quenchable colorimetric indicator 126 at 5′ end. Quenchable colorimetric indicator 126 can include a fluorescent indicator that may be detected using a suitable fluorescence detection method such as flow cytometry, fluorescence-activated cell sorting, FRET, and the like. Alternatively, quenchable colorimetric indicator 126 can include a dye or other visible color indicator that can be detected using a suitable colorimetric detector such as a spectrophotometer or a color activated cell sorting system. In some embodiments, suitable colorimetric indicators may include FAM, Tet M, Tet O, HEX, Cy3, TAMRA, ATTO, Cy5, and the like.

In some embodiments, bound strand 122 may include from about 1 nucleotide to about 30 nucleotides (e.g., 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides). In a preferred embodiment, bound strand 122 may include 21-24 nucleotides.

In some embodiments, bound strand 122 may include one or more locked nucleotides. In a preferred embodiment, bound strand 122 includes approximately 33% locked nucleotides. The locked nucleotides may be located at every 3rd base starting with the 3rd nucleotide from the immobilized end.

In some embodiments, bound strand 122 may include one or more abasic sites. In a preferred embodiment, bound strand 122 includes up to 20% abasic sites. The abasic sites may be located in the middle of the strand or may be located along the recognition domain 124 or the toehold domain 128.

As shown in FIG. 1, the soluble strand 132 of dsNA molecule 120 can contain a quencher 136 positioned at the 3′ end of the other strand 132. In some embodiments, quencher 136 can specifically quench colorimetric indicator 126 of bound strand 122 such as, for example, Black Hole and Iowa Black quenchers.

In some embodiments, soluble strand 132 may include from about 1 nucleotide to about 100 nucleotides (e.g., 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, or any number in between, e.g., 92 nucleotides). In a preferred embodiment, soluble strand 132 may include 24 nucleotides.

In some embodiments, the dsNA probes are specific to a virus or bacterium. Non-limiting examples of such viruses and bacteria include pathogenic viruses and bacteria, such as viruses and bacteria that are human pathogens. For instance, systems and methods described herein may be used to detect helical and envelope viruses in which RNA can be extracted into a sample. In some embodiments, the virus can be SARS-COVID-19. In some embodiments, the dsNA probes are specific for environmental contaminants (e.g., contaminants of water, wastewater, or water supplies), food contact surfaces, or food preparation contact surfaces, high contact surfaces such as door handles.

Any of the above embodiments of the dsNA probes may be used in any of the methods and/or kits described herein.

An exemplary kit according to some aspects of the invention can include a microsphere as described herein and dsNA probes as described herein. The dsNA probes can optionally be already bound to the microsphere surface. In some embodiments, the kit can be used to test for the presence of a certain nucleic acid in a sample. Non-limiting examples of samples that can be tested with kits according to the invention include biological samples, water samples, wastewater samples, environmental samples, food samples, and/or food contact surface samples, high contact surfaces areas such as door handles, bathrooms, faucet handles, computer keyboards, cell phones, stairway rails, elevator buttons, and the like. Non-limiting examples of biological samples include tissue, blood, serum, plasma, saliva, nasopharyngeal swab or secretion, urine, cerebrospinal fluid, lymphatic fluid, sputum, cell lysate and the like.

An exemplary method according to some aspects of the invention can include detecting and/or quantifying a nucleic acid of interest. In such a method, the nucleic acid can be incubated with a composition including any of the microspheres and dsNA probes described herein for a suitable amount of time and under suitable conditions for the nucleic acid to displace the soluble strand of the dsNA probe such that the colorimetric indicator is no longer quenched. After the incubation, the colorimetric indicator is measured by a suitable method, e.g., flow cytometry, FRET, spectrophotometry, etc., depending on the colorimetric indicator. The amount of fluorescence or other indicator enables the detection and optionally quantification of the nucleic acid of interest. In some embodiments, the nucleic acid can be isolated from a sample as described herein using appropriate techniques known in the art.

Another exemplary method according to some aspects of the invention can include detecting and/or quantifying a nucleic acid of interest in a biological sample from a subject. In such a method, the biological sample can be taken from the subject and the nucleic acid can be isolated from the biological sample using appropriate techniques known in the art. The isolated nucleic acid can then be incubated with a composition including any of the microspheres and dsNA probes described herein for a suitable amount of time and under suitable conditions for the nucleic acid to displace the soluble strand of the dsNA probe such that the colorimetric indicator is no longer quenched. After the incubation, the colorimetric indicator is measured by a suitable method, e.g., flow cytometry, FRET, spectrophotometry, etc., depending on the colorimetric indicator. The amount of fluorescence or other indicator enables the detection and optionally quantification of the nucleic acid of interest.

FIG. 1 illustrates displacement of the other strand 132 when a single stranded target NA molecule 142 hybridizes with the toehold region 128 of bound strand 122. In some embodiments, toehold domain 128 of the dsNA molecule can be designed to specifically match a single stranded NA molecule of a pathogen of interest. In such a case, a sample from a subject that includes such single stranded NA molecule would hybridize with bound strand 122 of composition 100 and displace the quencher of the colorimetric indicator 126 such that the colorimetric indicator is active (e.g., provide fluorescent signal). In some embodiments, strand 132 (the strand not bound to microsphere 110) can be soluble such that upon displacement of the strand 132 from bound strand 122, strand 132 is dissolved into the surroundings and unable to compete with the single stranded target NA molecule 142. Strand 132 is referred to herein as the “soluble strand”.

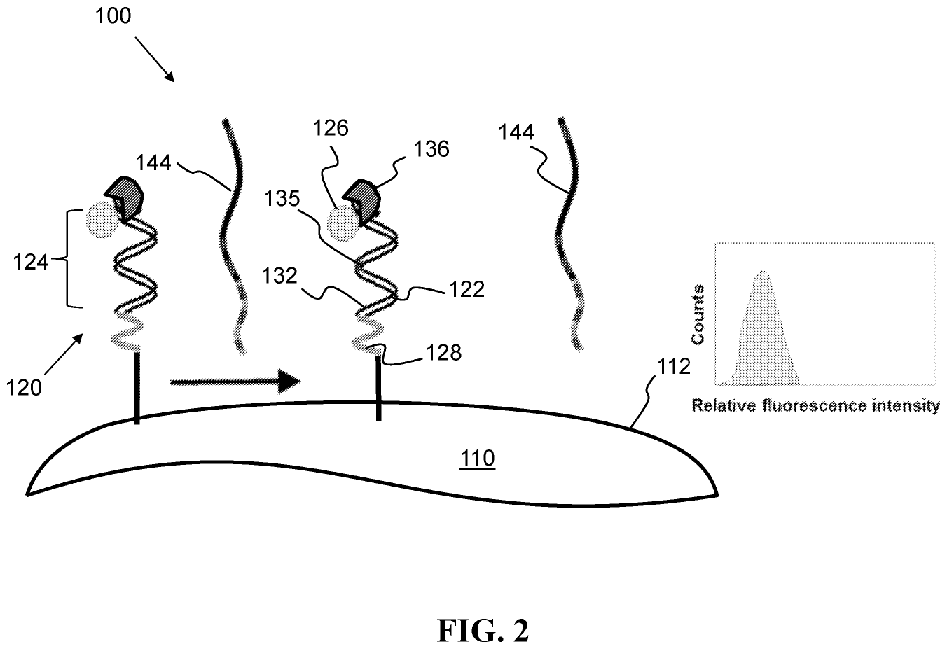

On the other hand, FIG. 2 illustrates no displacement of the soluble strand 132 when an imperfectly mismatched single stranded NA molecule 144 is introduced to composition 100 and fails to displace soluble strand 132 from bound strand 122, as indicated on the plot to the right. As demonstrated in FIG. 2, a sample that does not contain any single stranded NA molecule of a pathogen of interest would generate no signal.

In some embodiments, composition 100 may include two or more different dsNA sequences targeting a variety of pathogens (e.g., variants of a pathogen) such that when any of the single stranded NA molecules of a pathogen of interest are introduced to composition 100, one or more soluble strands 132 may be displaced by the target NA molecule 142, indicating the presence of one or more variants of the pathogen of interest.

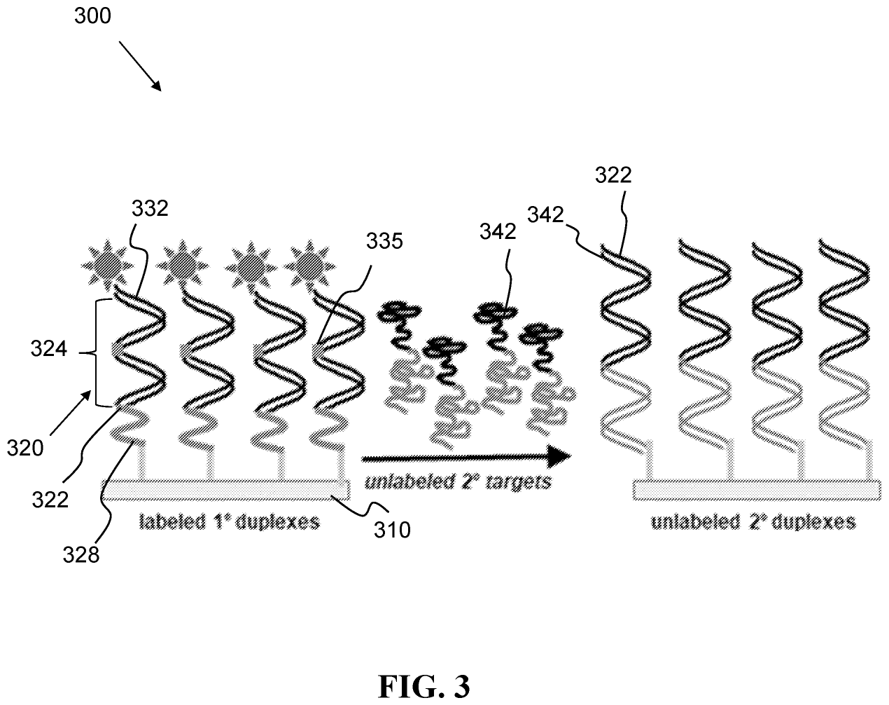

In some embodiments, composition 100 can further include a mismatched base pair 135, shown more clearly in FIGS. 2 and 3. Mismatched base pair 135 between bound strand 122 and soluble strand 132 can be positioned in the recognition domain 124 of dsNA molecule 120. In addition, or alternatively thereto, mismatched base pair 135 may be positioned in the toehold domain 128 of bound strand 122. In some embodiments, mismatched base pair 135 may be positioned in both the recognition domain 124 and the toehold domain 128.

In any of the embodiments disclosed herein, the bound strand 122 of the dsNA molecule 120 can include a COVID-19-specific nucleic acid. For instance, the bound strand 122 can encode a COVID-19-specific protein. In particular, the bound strand can include a sequence selected from the group provided in Table 1.

TABLE 1
Sequence and nomenclature of LNA, DNA, and RNA oligonucleotides in example
dsNA molecules, where bold, underlined, and italicized portions represent toehold
domains; X represents abasic nucleotide in select quencher-capped hybridization partners;
B = C, G, or T; D = A, G, or T; and H = A, C, or T in a mixture of model RNA sequence
variants to SARS-CoV-2 RNA. Nucleotides followed by a superscript “L” are
locked nucleotides.

| Sequence | Nomenclature |
|---|---|
| 5′-FAM-ACC^{L}CCG^{L}CAT^{L}TAC^{L}GTT^{L}TGG^{L}TGG^{L}ACC^{L}-biotin-3′ | LNA N1-FAM |
| (SEQ ID NO: 1) | |
| 3′-Q-TGG^{L}GGC^{L}GTA^{L}ATG^{L}CAA^{L}ACC^{L}ACC^{L}-5′ (SEQ ID NO: 2) | LNA 21Q |
| 3′-Q-TGG^{L}GGC^{L}GTA^{L}ATG^{L}CAA^{L}-5′ (SEQ ID NO: 3) | LNA 15Q |
| 3′-Q-TGG^{L}GGC^{L}GTA^{L}-5′ (SEQ ID NO: 4) | LNA 9Q |
| 3′-Q-TGG^{L}GGC^{L}GTA^{L}AXG^{L}CAA^{L}ACC^{L}ACC^{L}-5′ (SEQ ID NO: 5) | LNA 21mQ |
| 3′-Q-TGG^{L}GGC^{L}GXA^{L}ATG^{L}CAA^{L}-5′ (SEQ ID NO: 6) | LNA 15mQ |
| 3′-Q-TGG^{L}GXC^{L}GTA^{L}-5′ (SEQ ID NO: 7) | LNA 9mQ |
| 5′-FAM-ACC CCG CAT TAC GTT TGG TGG ACC-biotin-3′ | N1-FAM |
| (SEQ ID NO: 12) | |
| 3′-Q-TGG GGC GTA ATG CAA ACC ACC-5′ (SEQ ID NO: 13) | 21Q |
| 3′-Q-TGG GGC GTA ATG CAA-5′ (SEQ ID NO: 14) | 15Q |
| 3′-Q-TGG GGC GTA-5′ (SEQ ID NO: 15) | 9Q |
| 3′-Q-TGG GGC GTA AXG CAA ACC ACC-5′ (SEQ ID NO: 8) | 21mQ |
| 3′-Q-TGG GGC GXA ATG CAA-5′ (SEQ ID NO: 9) | 15mQ |
| 3′-Q-TGG GGC XTA ATG C-5′ (SEQ ID NO: 10) | 13mQ |
| 3′-Q-TGG GGX GTA AT-5′ (SEQ ID NO: 16) | 11mQ |
| 3′-Q-TGG GXC GTA-5′ (SEQ ID NO: 11) | 9mQ |
| 3′-UGG GGC GUA AUG CAA ACC ACC UGG-5′ | SARS-CoV-2 RNA |
| (SEQ ID NO: 23) | |
| 3′-GCC AUG ACC GGU CAU AGU ACG GAG-5′ | scr_RNA |
| (SEQ ID NO: 24) | |
| 3′-UGG GGC GUA AUH CAB ACD ACD UGH-5′ | var_RNA |
| (SEQ ID NO: 25) | |

In any of the embodiments disclosed herein, a composition 300 can be quantitatively determined by measuring the extent of displacement activity in a soluble strand 332. FIG. 3 provides a schematic of such quantitative assessments. As shown, composition 300 can be initially fluorescent primary dsNA 320 with an unlabeled strand 322 immobilized on a microsphere 310 or particle as described supra. The unlabeled strand 322 can be hybridized to FAM-labeled 15m hybridization strand 332 that can be designed to be selectively susceptible to displacement by an unlabeled target 342. Upon displacement of the hybridization strand 332, composition 300 is capable of forming nonfluorescent secondary duplexes 400. In a similar mechanism as described supra, primary dsNA 320 may include a recognition domain 324 and a toehold domain 328 on the immobilized unlabeled strand 322. In some embodiments, composition 300 may include a fluorescent tag on the soluble/hybridization strand 332 that is not quenchable. In addition, composition 300 may further lack a quencher on the immobilized strand 322. Upon introduction of an unlabeled target 342 such as a pathogen RNA (e.g., such as SARS-COV-2 RNA), the dsNA 320 undergoes a disassociation of the fluorescent soluble strand 332 such that the composition 300 is no longer providing a signal that can be correlated into a quantitative assessment of the displacement activity. The displacement activity of composition 300 may be adjusted by adding one or more mismatched base pairs 335 between the immobilized strand 322 and the fluorescent soluble strand 332.

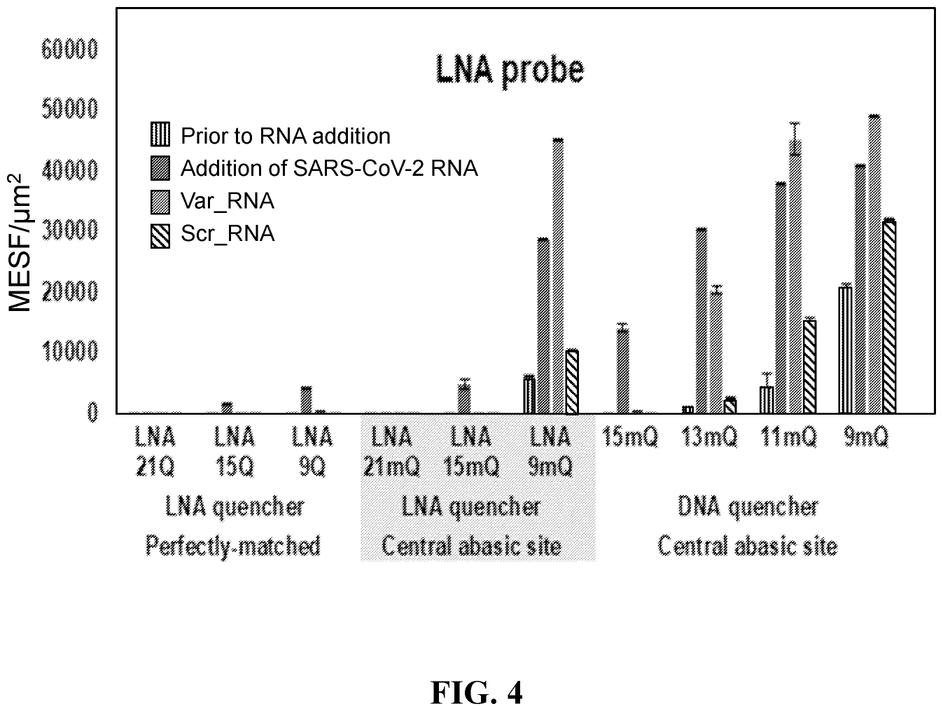

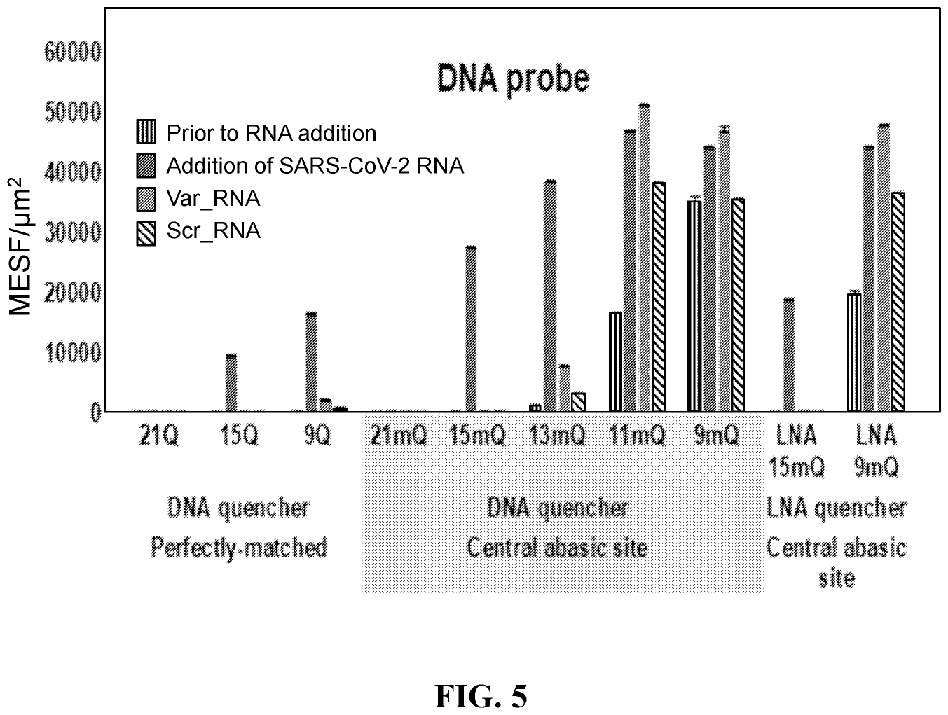

As shown in FIGS. 4 and 5, plots of displacement activity for example compositions 100 are provided with LNA or DNA dsNA molecules 120 of varying length and number of mismatches. When LNA or DNA dsNA molecules 120 are hybridized with perfectly-matched sequence pairs (e.g., “LNA XQ” or “DNA XQ”), the target sequence is unable to compete and little to no fluorescence is produced unless the length of the LNA or DNA dsNA molecule is very short (e.g., 9 bases long). Alternatively, LNA or DNA dsNA molecules 120 having a central abasic site (e.g., “LNA XmQ” or “DNA “XmQ”) exhibited fluorescence signals when introduced to a sample including a target single stranded NA molecule 142 (e.g., SARS-COV-2 RNA). Although with a short sequence such as 9 bases or less, the soluble strand 132 may be displaced even without any target single stranded NA molecules. As shown, preferred LNA dsNA molecules 120 present fluorescent signals only after the addition of a target of interest, such as SARS-COV-2 RNA. As shown, 15nQ presents a desirable balance of stability and responsiveness due to neighboring 6 nucleotide toehold for 24 base-long target with a central abasic site.

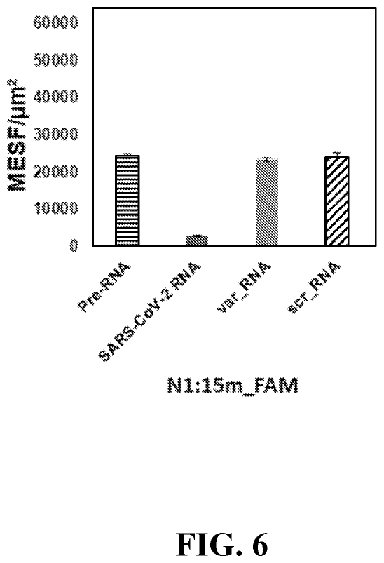

FIG. 6 provides quantitative data of displacement activity of composition 300. An example composition 300 including N1:15m_FAM primary dsNA duplexes 320 prior to addition of any unlabeled targets 342, after the addition of an unlabeled targets 342 (e.g., SARS-COV-2 RNA), after the addition of an unlabeled mismatched target 344 (e.g., var_RNA or scr_RNA sequence).

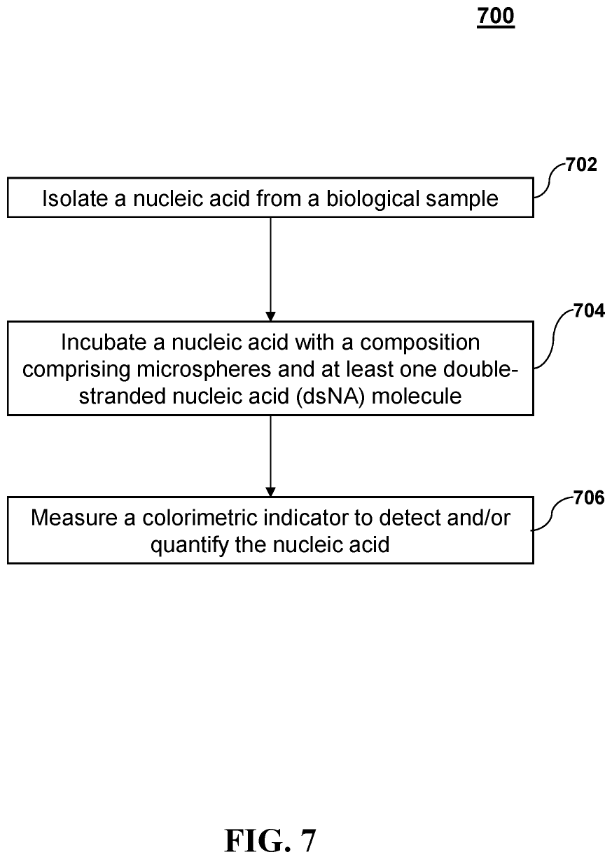

FIG. 7 is a flow-chart of a method 700 for making nucleic acid detection platforms 100. Method 700 can include isolating 702 a nucleic acid from a biological sample. Method 700 can also include incubating 704 a nucleic acid with a composition 100 that includes microspheres 110 and at least one double-stranded nucleic acid (dsNA) molecule 120. One strand 122 of the dsNA molecule 120 can be bound, at 3′ end of the strand, to an outer surface 112 of the microspheres 120. The bound strand 122 of the dsNA molecule 120 can include a recognition domain 124. The bound strand 122 can also contain a quenchable colorimetric indicator 126 at a 5′ end. The other strand 132 of the dsNA molecule 120 can be soluble and contain a quencher of the colorimetric indicator 136 at a 3′ end. The soluble strand 132 can be configured to be displaced by the nucleic acid 142, thereby displacing the quencher of the colorimetric indicator such that the colorimetric indicator is active. Method 700 can further include measuring 706 the colorimetric indicator to detect and/or quantify the nucleic acid 142.

The following examples further illustrate aspects of the present disclosure. However, they are in no way a limitation of the teachings or disclosure of the present disclosure as set forth herein.

### EXAMPLES

In an example experimentation, a series of 20 candidate locked nucleic acid (LNA) and DNA dsprobes were immobilized on microspheres which were incubated briefly with various RNA sequences, and then quickly interrogated using flow cytometry. While clear and surprising trends in stability and RNA target responsiveness are evident across the sequence parameter space explored here, certain DNA dsprobe systems emerged as highly stable, yet specifically responsive to SARS-COV-2 RNA.

### Example 1—Materials and Methods

All DNA, LNA, and RNA sequences were purchased from Integrated DNA Technologies (Coralville, IA), with standard desalting. For data in FIGS. 4 and 5, each immobilized probe possessed a 6-carboxyfluorescein (FAM) moiety at the 5′ end. Each hybridization partner possessed an Iowa Black® Fluorescence Quencher (Q) moiety on 3′ end. Stock oligonucleotides solutions were prepared and stored in TE pH 8.0 (Sigma Aldrich, St. Louis, MO) at 100 μM. Prior to coupling to microspheres, probes and their hybridization partners were annealed by heating the solutions (2:1 quencher-capped hybridization partner to FAM-labeled biotinylated probes) to 94° C. for 2 min, then slowly cooling to room temperature. For microsphere coupling, working suspensions of 1.05 μm dia. streptavidin-coated microspheres (Bangs Laboratory, Fishers, IN) were prepared by diluting stock from 1% w/v to 0.1% w/v in wash buffer (20 mM Tris (Sigma Aldrich), 1 M NaCl (Sigma Aldrich), 1 mM EDTA (Promega, Madison, WI), 0.0005% Triton X-100 (Sigma Aldrich)), per Bangs Product Data Sheet 721. Prior to oligonucleotide coupling, microspheres were washed 3 times by centrifuging at 14,000 g for 3 min, aspirating supernatant, and resuspending in wash buffer. The pre-annealed solutions of the dsprobe systems were added to washed microspheres for a final probe concentration of 5 μM and agitated for 15 minutes at 22° C., 800 rpm on a thermomixer. Following incubation, suspension samples were washed 3 times as described above. For displacement experiments, RNA stock was added to the washed and dsprobe-functionalized microspheres for final concentration of 10 μM RNA and agitated for 15 minutes at 22° C., 800 rpm on a thermomixer. Microspheres were not washed following RNA addition. To prepare samples for flow cytometry, a 5 μL microsphere suspension aliquot was added to 1000 μL PBS (Gibco, Waltham, MA). Singlet populations gated based on size, and 10,000 counts recorded. Molecules of equivalent soluble fluorochrome (MESF) was calculated from raw voltage using Bangs Laboratory Quantum FITC-5 MESF standard beads. The average MESF/μm² and standard error are reported for three suspensions prepared for each dsprobe system. MESF values were normalized to the degree of labeling for each fluorescent sequence, calculated using spectrophotometry data.

### Example 2—Synthesis of dsprobes

Example candidate dsprobe systems is provided in Table 2 (below). Each dsprobe includes a biotinylated, 24 base-long 6-carboxyfluorescein (FAM)-tagged probe (i.e., LNA N1_FAM or N1_FAM) that is perfectly complementary to the 24 base-long RNA target (i.e., SARS-COV-2 RNA). To form dsprobes in an initially signal-off state, probes can be incubated with 1 of 20 Iowa Black® Fluorescence Quencher (Q)-capped hybridization partners of varying lengths (i.e., 9 to 21 bases), chemical modifications (i.e., locked vs. natural deoxyribose) and sequence fidelity (e.g., 8 hybridization partners possess a central, non-hybridizing abasic site) and then conjugated to streptavidin-coated microspheres as detailed in the Materials and Methods Section. For simplicity, any LNA-DNA chimera will be referred to as an LNA sequence. Consistent with displacement-based strategies9-11, next to the pre-hybridized recognition segment illustrated in FIG. 1A is a single-stranded or initially unhybridized segment ranging from 4 to 15 bases in length in each dsprobe. This single-stranded segment is intended to serve as a toehold for SARS-COV-2 RNA to initiate duplex formation.

Table 2 provides example sequences and nomenclature of LNA, DNA, and RNA oligonucleotides employed in which the superscript “L” indicates a locked nucleotide in select FAM-functionalized probe and quencher-capped hybridization sequences; X=abasic nucleotide in select quencher-capped hybridization partners; B=C, G, or T; D=A, G, or T; and H=A, C, or T in a mixture of model RNA sequence variants to SARS-COV-2 RNA. Each dsprobe is comprised of a 5′ FAM moiety on the immobilized sequence and a 3′ quencher (Q) on its hybridization partner. The choice of DNA probe sequence is identical to the probe sequence named 2019-nCOV_N1-P on the Centers for Disease Control website (cdc.gov/coronavirus/2019-ncov/lab/rt-pcr-panel-primer-probes.html) accessed on Feb. 11, 2022.

TABLE 2
Sequence, nomenclature, and function of LNA, DNA, and RNA oligonucleotides in
example dsNA molecules; where bold, underlined, and italicized portions represent toehold
domains; X represents abasic nucleotide in select quencher-capped hybridization partners; B
= C, G, or T; D = A, G, or T; and H = A, C, or T in a mixture of model RNA sequence
variants to SARS-CoV-2 RNA.

| Sequence | Nomenclature | Function |
|---|---|---|
| 5′-FAM-ACC^{L}CCG^{L}CAT^{L}TAC^{L}GTT^{L}TGG^{L}TGG^{L}ACC^{L}-biotin-3′ | LNA N1- | LNA:LNA |
| (SEQ ID NO: 1) | FAM:LNA 21Q | dsprobes |
| 3′-Q-TGG^{L}GGC^{L}GTA^{L}ATG^{L}CAA^{L}ACC^{L}ACC^{L}-5′ (SEQ ID NO: 2) | | (perfectly |
| 5′-FAM-ACC^{L}CCG^{L}CAT^{L}TAC^{L}GTT^{L}TGG^{L}TGG^{L}ACC^{L}-biotin-3′ | LNA N1- | matched) |
| (SEQ ID NO: 1) | FAM:LNA 15Q | |
| 3′-Q-TGG^{L}GGC^{L}GTA^{L}ATG^{L}CAA^{L}-5′ (SEQ ID NO: 3) | | |
| 5′-FAM-ACC^{L}CCG^{L}CAT^{L}TAC^{L}GTT^{L}TGG^{L}TGG^{L}ACC^{L}-biotin-3′ | LNA N1- | |
| (SEQ ID NO: 1) | FAM:LNA 9Q | |
| 3′-Q-TGG^{L}GGC^{L}GTA^{L}-5′ (SEQ ID NO: 4) | | |
| 5′-FAM-ACC^{L}CCG^{L}CAT^{L}TAC^{L}GTT^{L}TGG^{L}TGG^{L}ACC^{L}-biotin-3′ | LNA N1- | LNA:LNA |
| (SEQ ID NO: 1) | FAM:LNA | dsprobes |
| 3′-Q-TGG^{L}GGC^{L}GTA^{L}AXG^{L}CAA^{L}ACC^{L}ACC^{L}-5′ (SEQ ID NO: 5) | 21 mQ | (with central |
| 5′-FAM-ACC^{L}CCG^{L}CAT^{L}TAC^{L}GTT^{L}TGG^{L}TGG^{L}ACC^{L}-biotin-3′ | LNA N1- | abasic site) |
| (SEQ ID NO: 1) | FAM:LNA | |
| 3′-Q-TGG^{L}GGC^{L}GXA^{L}ATG^{L}CAA^{L}-5′ (SEQ ID NO: 6) | 15mQ | |
| 5′-FAM-ACC^{L}CCG^{L}CAT^{L}TAC^{L}GTT^{L}TGG^{L}TGG^{L}ACC^{L}-biotin-3′ | LNA N1- | |
| (SEQ ID NO: 1) | FAM:LNA 9mQ | |
| 3′-Q-TGG^{L}GXC^{L}GTA^{L}-5′ (SEQ ID NO: 7) | | |
| 5′-FAM-ACC^{L}CCG^{L}CAT^{L}TAC^{L}GTT^{L}TGG^{L}TGG^{L}ACC^{L}-biotin-3′ | LNA N1- | LNA:DNA |
| (SEQ ID NO: 1) | FAM: 21mQ | dsprobes |
| 3′-Q-TGG GGC GTA AXG CAA ACC ACC-5′ (SEQ ID NO: 8) | | (with central |
| 5′-FAM-ACC^{L}CCG^{L}CAT^{L}TAC^{L}GTT^{L}TGG^{L}TGG^{L}ACC^{L}-biotin-3′ | LNA N1- | abasic site) |
| (SEQ ID NO: 1) | FAM: 15mQ | |
| 3′-Q-TGG GGC GXA ATG CAA^{L}-5′ (SEQ ID NO: 9) | | |
| 5′-FAM-ACC^{L}CCG^{L}CAT^{L}TAC^{L}GTT^{L}TGG^{L}TGG^{L}ACC^{L}-biotin-3′ | LNA N1- | |
| (SEQ ID NO: 1) | FAM:13mQ | |
| 3′-Q-TGG GGC XTA ATG C-5′ (SEQ ID NO: 10) | | |
| 5′-FAM-ACC^{L}CCG^{L}CAT^{L}TAC^{L}GTT^{L}TGG^{L}TGG^{L}ACC^{L}-biotin-3′ | LNA N1- | |
| (SEQ ID NO: 1) | FAM:9mQ | |
| 3′-Q-TGG GXC GTA -5′ (SEQ ID NO: 11) | | |
| 5′-FAM-ACC CCG CAT TAC GTT TGG TGG ACC-biotin-3′ | N1-FAM:21Q | DNA:DNA |
| (SEQ ID NO: 12) | | dsprobes |
| 3′-Q-TGG GGC GTA ATG CAA ACC ACC-5′ (SEQ ID NO: 13) | | (perfectly- |
| 5′-FAM-ACC CCG CAT TAC GTT TGG TGG ACC-biotin-3′ | N1-FAM:15Q | matched) |
| (SEQ ID NO: 12) | | |
| 3′-Q-TGG GGC GTA ATG CAA-5′ (SEQ ID NO: 14) | | |
| 5′-FAM-ACC CCG CAT TAC GTT TGG TGG ACC-biotin-3′ | N1-FAM:9Q | |
| (SEQ ID NO: 12 | | |
| 3′-Q-TGG GGC GTA-5′ (SEQ ID NO: 15) | | |
| 5′-FAM-ACC CCG CAT TAC GTT TGG TGG ACC-biotin-3′ | N1-FAM:21mQ | DNA:DNA |
| (SEQ ID NO: 12) | | dsprobes |
| 3′-Q-TGG GGC GTA AXG CAA ACC ACC-5′ (SEQ ID NO: 8) | | (with central |
| 5′-FAM-ACC CCG CAT TAC GTT TGG TGG ACC-biotin-3′ | N1-FAM: 15mQ | abasic site) |
| (SEQ ID NO: 12) | | |
| 3′-Q-TGG GGC GXA ATG CAA-5′ (SEQ ID NO: 9) | | |
| 5′-FAM-ACC CCG CAT TAC GTT TGG TGG ACC-biotin-3′ | N1-FAM:13mQ | |
| (SEQ ID NO: 12) | | |
| 3′-Q-TGG GGC XTA ATG C-5′ (SEQ ID NO: 10) | | |
| 5′-FAM-ACC CCG CAT TAC GTT TGG TGG ACC-biotin-3′ | N1-FAM:11mQ | |
| (SEQ ID NO: 12) | | |
| 3′-Q-TGG GGX GTA AT-5′ (SEQ ID NO: 16) | | |
| 5′-FAM-ACC CCG CAT TAC GTT TGG TGG ACC-biotin-3′ | N1-FAM: 9mQ | |
| (SEQ ID NO: 12) | | |
| 3′-Q-TGG GXC GTA-5′ (SEQ ID NO: 11) | | |
| 5′-FAM-ACC CCG CAT TAC GTT TGG TGG ACC-biotin-3′ | N1-FAM:LNA | DNA:LNA |
| (SEQ ID NO: 12) | 15mQ | dsprobes |
| 3′-Q-TGG^{L}GGC^{L}GXA^{L}ATG^{L}CAA^{L}-5′ (SEQ ID NO: 6) | | (with central |
| 5′-FAM-ACC CCG CAT TAC GTT TGG TGG ACC-biotin-3′ | N1-FAM:LNA | abasic site) |
| (SEQ ID NO: 12) | 9mQ | |
| 3′-Q-TGG^{L}GXC^{L}GTA^{L}-5′ (SEQ ID NO: 7) | | |
| 5′-FAM-ACC^{L}CCG^{L}CAT^{L}TAC^{L}GTT^{L}TGG^{L}TGG^{L}ACC^{L}-biotin-3′ | N1_Hybridization | |
| (SEQ ID NO: 1) | Partner2 | |
| 3′-Q-TGG^{L}GGC^{L}GTA^{L}ATG^{L}CAA^{L}ACC^{L}-5′ (SEQ ID NO: 17) | | |
| 5′-FAM-ACC^{L}CCG^{L}CAT^{L}TAC^{L}GTT^{L}TGG^{L}TGG^{L}ACC^{L}-biotin-3′ | N1_mismatched | |
| (SEQ ID NO: 1) | Hyb Partner2m | |
| 3′-Q-TGG^{L}GGC^{L}GTA^{L}XTG^{L}CAA^{L}ACC^{L}-5′ (SEQ ID NO: 18) | | |
| 5′-FAM-ACC^{L}CCG^{L}CAT^{L}TAC^{L}GTT^{L}TGG^{L}TGG^{L}ACC^{L}-biotin-3′ | N1_Hybridization | |
| (SEQ ID NO: 1) | Partner4 | |
| 3′-Q-TGG^{L}GGC^{L}GTA^{L}ATG^{L}-5′ (SEQ ID NO: 19) | | |
| 5′-FAM-ACC^{L}CCG^{L}CAT^{L}TAC^{L}GTT^{L}TGG^{L}TGG^{L}ACC^{L}-biotin-3′ | N1_mismatched | |
| (SEQ ID NO: 1) | Hyb Partner4m | |
| 3′-Q-TGG^{L}GGC^{L}XTA^{L}ATG^{L}-5′ (SEQ ID NO: 20) | | |
| 5′-FAM-ACA^{L}ATT^{L}TGC^{L}CCC^{L}CAG^{L}CGC^{L}TTC^{L}AG-biotin-3′ | 2019-nCoV_N2 | |
| (SEQ ID NO: 21) | microsphere- | |
| | immobilized | |
| | sequence | |
| 3′-Q-TGT^{L}TAA^{L}ACG^{L}GGG^{L}GTC^{L}GC-5′ (SEQ ID NO: 22) | N2_Hybridization | |
| | Partner1 | |
| 3′-UGG GGC GUA AUG CAA ACC ACC UGG-5′ (SEQ ID NO: 23) | SARS-CoV-2 | RNA target |
| | RNA | |
| 3′-GCC AUG ACC GGU CAU AGU ACG GAG-5′ (SEQ ID NO: 24) | scr_RNA | Negative |
| 3′-UGG GGC GUA AUH CAB ACD ACD UGH-5′ (SEQ ID NO: 25) | var_RNA | RNA targets |

To complete duplex formation with the recognition segment, the unlabeled RNA target must fully displace the quencher-capped LNA or DNA hybridization partner. A fluorescence signal from the now unquenched N1_FAM should ensue from a successful toehold-mediated displacement event. To assess target specificity, a heterogeneous mixture of ˜243 similar, yet imperfectly-matched sequences (i.e., var_RNA) is included (see Table 2). Depending on the base-length and fidelity of the quencher-capped hybridization partner, one or more mismatches occur within each var_RNA segment intended to first hybridize to the toehold segment of the immobilized probe. Thus, as prior oligonucleotide solution experiments and theoretical studies indicate, though partially mismatched with respect to the microsphere-immobilized probe, the greater number of total base-pair matches in the recognition+toehold segments may still favor displacement activity of any shorter, quencher-capped hybridization partner. In contrast, the scrambled RNA sequence (i.e., scr_RNA) possesses only a few Watson-Crick base-pair matches with the probe and is thus not anticipated to participate in toehold-mediated displacement events.

The first dsprobe candidates consisted of LNA N1_FAM and quencher-capped LNA hybridization partners of three different lengths: 21 bases (i.e., LNA 21Q), 15 bases (i.e., LNA 15Q), and 9 bases (i.e., LNA 9Q). As shown in FIG. 4, these three dsprobe systems all exhibited little to no fluorescence signal prior to SARS-COV-2 RNA addition as well in the presence of any noncomplementary RNA. Following the addition of SARS-COV-2 RNA, the longest 21 base-long LNA dsprobe showed no increase in fluorescence while the 15 base and 9 base-long LNA dsprobes showed only modest increases in fluorescence indicating little displacement by SARS-COV-2 RNA occurs in these perfectly-matched LNA dsprobes. To promote additional selective displacement activity while maintaining stability, these studies were repeated using analogous quencher-capped LNA hybridization partners with a central base missing (i.e., abasic site). Probes initially hybridized to LNA 21 mQ exhibited the same lack of fluorescence activity in the both the absence and presence of any RNA sequences. In contrast, a relatively high background fluorescence signal was found for LNA 9 mQ indicating fewer dsprobes formed initially. While fluorescence further increased upon the addition of SARS-COV-2 RNA, the increase in fluorescence also occurred in the presence of noncomplementary RNA. The most promising LNA dsprobe system employed LNA 15 mQ as it allowed for fluorescence signaling only in the presence of SARS-COV-2 RNA; however, its observed fluorescence was significantly lower than that of the shortest mismatched dsprobe in the presence of SARS-COV-2 RNA (i.e., LNA N1_FAM: LNA 9 mQ+SARS-COV-2). Thus, exchange of quencher-capped LNA 15 mQ with SARS-COV-2 RNA appears incomplete. Overall, with the exception of LNA N1_FAM: LNA 9 mQ exhibiting both higher background signal and less RNA target specificity, the affinity of LNA: LNA duplexes in the dsprobes explored here appears too high to be susceptible to extensive displacement by SARS-COV-2 RNA.

Since DNA is a reportedly weaker hybridization partner compared to LNA, the stability and responsiveness of LNA N1_FAM hybridized to quencher-capped 9, 11, 13, and 15 base-long DNA with a central abasic site were also examined. For all but the longest of these DNA hybridization partners (i.e. 15 mQ), the background fluorescence signal and fluorescence response to negative RNA targets otherwise increased as the DNA hybridization partner base length decreased as shown in FIG. 4 for 13 mQ, 11 mQ, and 9 mQ, respectively. While the fluorescence signal for LNA N1_FAM:15 mQ dsprobes is nearly double that of LNA N1_FAM: LNA 15 mQ dsprobes in the presence of SARS-COV-2 RNA, it is still well below the fluorescence signal of all the remaining dsprobes possessing shorter, mismatched DNA hybridization partners (i.e., 13 mQ, 11 mQ, and 9 mQ) in the presence of SARS-COV-2. Thus, while selective displacement activity appears enhanced by replacing the quencher-capped, 15 base-long, central abasic LNA hybridization partner with its equivalent DNA sequence, further improvements in detecting SARS-COV-2 RNA appeared possible with stable, yet weaker affinity DNA: DNA sequence pairings in dsprobes.

Next, pure DNA dsprobes comprised of N1_FAM and various quencher-capped DNA hybridization partners were examined. As before with LNA dsprobes, first the background fluorescence signal is measured and determined to be negligible for all perfectly-matched DNA dsprobes in FIG. 5. In comparison to the weaker fluorescence activity of perfectly-matched LNA dsprobe analogues in FIG. 4 in the presence of SARS-COV-2 RNA, a stronger dependence of fluorescence signaling on base length of 21Q, 15Q, and 9Q in the presence of SARS-COV-2 RNA is evident in FIG. 5. To further explore the ability to further improve SARS-COV-2 RNA detection without compromising dsprobe stability, a series of quencher-capped DNA hybridization partners with a central abasic site was then explored. Unlike the perfectly-matched dsprobes, however, a trend of increasing background fluorescence in the absence of RNA is observable for 13 mQ, 11 mQ, and 9 mQ, respectively indicating this series of imperfectly-matched dsprobes is increasingly susceptible to spontaneous dissociation. Thus, while additional fluorescence activity in the presence of SARS-COV-2 RNA does occur in each of these three imperfectly-matched dsprobe system in FIG. 5, their lower stability broadens their susceptibility to either dissociation (likely followed by RNA hybridization to unoccupied N1_FAM) or to displacement by var_RNA sequences that resemble, but do not entirely match SARS-COV-2 RNA sequences. LNA 15 mQ and LNA 9 mQ as hybridization partners to this DNA probe in FIG. 5 showed similar fluorescence behavior as their sequence counterparts in FIG. 4 (i.e., LNA N1_FAM:15 mQ and LNA N1_FAM:9 mQ, respectively) Thus, along with all the LNA:LNA, LNA:DNA, and perfectly-matched DNA:DNA dsprobe systems discussed earlier, DNA probes initially hybridized to 13 mQ, 11 mQ, 9 mQ, LNA 15 mQ, or LNA 9 mQ were not further considered as optimal candidates.

In contrast to dsprobe systems discussed thus far, one particular DNA dsprobe system, namely, N1_FAM:15 mQ, exhibited the best balance as a stable dsprobe that is also highly and selectively responsive to SARS-COV-2 RNA. As shown in FIG. 5 this 15 base-long hybridization partner with a central abasic site forms an imperfect (i.e., one symmetric internal loop flanked by two 7 base-long duplexes), yet sufficiently stable dsprobe with a 9 base-long toehold that allows for extensive, yet selective displacement of 15 mQ by SARS-CoV-2 RNA to induce substantial fluorescence from N1_FAM. Intriguingly, members of the heterogeneous RNA mixture do not appear competitive as replacements for 15 mQ despite each var_RNA sequence having more total base-pair matches than 15 mQ for N1_FAM probe including a continuous 11 base-long match to the recognition segment and multiple, though noncontinuous base-pair matches for the remainder of the recognition segment and adjacent toehold segment as indicated in Table 3.

TABLE 3
Sequence and nomenclature of DNA and RNA oligonucleotides in example dsNA
molecules; where bold, underlined, and italicized portions represent toehold
domains; X represents abasic nucleotide in select quencher-capped hybridization
partners; B = C, G, or T; D = A, G, or T; and H = A, C, or T in a mixture of
model RNA sequence variants to SARS-CoV-2 RNA.

| Sequence | Nomenclature |
|---|---|
| 5′-ACCCCGCATTACGTTTGGTGGACC-3′ | N1_FAM: |
| 3′-TGGGGCGXAATGCAA-5′ | 15mQ |
| 3′-UGGGGCGUAAUHCABACDACDUGH-5′ | var_RNA |
| 3′-UGGGGCGUAAUGCAAACCACCUGG-5′ | SARS-CoV-2 RNA |
| 3′-GCCAUGACCGGUCAUAGUACGGAG-5′ | scr_RNA |

The dsprobe possesses a 5′ FAM moiety on the immobilized sequence and a 3′ quencher (Q) on its hybridization partner. To facilitate comparison to the quencher-capped hybridization partner in the dsprobe, all RNA sequences are shown 3′-+5′.

To quantitatively assess the extent of displacement activity in the N1:15m dsprobe system, separate experiments were conducted with unlabeled N1 sequences immobilized on microspheres and initially hybridized to FAM-tagged 15m hybridization partners to form fluorescent primary duplexes as illustrated in FIG. 3 (left). In contrast to dsprobes initially in a quenched state in FIGS. 1 and 2, the lack of any quencher species in N1:15m_FAM primary duplexes allows fluorescence events to occur until an event drives removal of the soluble 15m_FAM sequence. To thus quantify the amount of 15m_FAM sequences removed in the presence of various RNA sequence, the duplex density is measured before RNA addition, then after adding scr_RNA (to assess simple dissociation events) and var_RNA (to assess displacement by imperfectly-matched RNA). As shown in FIG. 6, less than 4% decrease in duplex densities occurs with either of these RNA controls. In contrast, the primary duplex density decreases by 88% in the presence of SARS-COV-2 RNA. Based on the overall stability of N1:15m_FAM in the presence of noncomplementary or imperfectly-matched RNA, this marked decrease in the presence of SARS-COV-2 RNA must be due to displacement of the shorter, FAM-labelled sequence with a central abasic site as illustrated in FIG. 3 (right).

This successful study efficiently screened numerous sequence combinations of modified and natural oligonucleotides to identify suitable dsprobes for unlabeled RNA detection using minimal chemical reagents, material supplies, and handling steps. Each suspension sample required ˜30 min handling time (i.e., dsprobe immobilization on microspheres followed by RNA incubation) with subsequent flow cytometry analysis requiring less than 1 min. In addition to the inherent flexibility of this detection platform to redesign and rapidly screen numerous dsprobe candidates to accommodate emerging variants or even new unrelated targets, one can also combine parallel processing of suspensions with modern flow cytometers capable of automated multi-well data collection. Moreover, the capabilities of this platform can be further expanded to both capture and separate targets via FACS-based sorting (i.e., separate fluorescent from quenched probe-functionalized microspheres) for further analysis (e.g., sequencing) as warranted. Such endeavors present exciting pathways for rapid and facile implementation of dsprobes for target detection and capture as part of population surveillance such as wastewater testing where timely results are essential.

### Example 3—Exemplary Isolation of Nucleic Acid from a Sample

In an example, a sample is obtained. Non-limiting examples of the sample include a biological sample, a water sample, a wastewater sample, an environmental sample, a food sample, a food contact surface sample, and/or a high surface contact area sample. Non-limiting examples of biological samples include tissue, blood, serum, plasma, saliva, nasopharyngeal swab or secretion, urine, cerebrospinal fluid, lymphatic fluid, sputum, cell lysate, and the like.

A nucleic acid is isolated from the sample using conventional nucleic acid isolating techniques. The isolated nucleic acid is then incubated with a microsphere comprising a dsprobe according to any of the embodiments described herein under conditions suitable for the isolated nucleic acid to displace the soluble strand of the dsprobe. The bound strand of the dsprobe is then able to fluoresce. The fluorescence can be detected and/or quantitated by any conventional methods, such as for example and not limitation, a high-throughput spectrophotometer, a fluorescence activated cell sorting (FACS) system, flow cytometer, or a color activated cell sorting system.

### LIST OF EMBODIMENTS

A non-exhaustive list of embodiments contemplated by the invention follows.

1. A composition comprising:
microspheres; and
at least one double-stranded nucleic acid (dsNA) molecule,
wherein one strand of the at least one dsNA molecule is bound to an outer surface of the microspheres at a 3′end,
wherein the bound strand of the at least one dsNA molecule comprises a recognition domain and contains a quenchable colorimetric indicator at a 5′ end, and
wherein the other strand of the at least one dsNA molecule is soluble and contains a quencher of the colorimetric indicator at a 3′ end.

2. The composition of item 1, wherein the microspheres are coated in streptavidin and the bound strand of the at least one dsNA molecule comprises biotin at 3′ end.

3. The composition of item 1 or 2, wherein the bound strand of the at least one dsNA molecule further comprises a toehold domain at 3′ end.

4. The composition of item 3, wherein the toehold domain comprises between 1 and 12 nucleotides.

5. The composition of items 3 or 4, wherein there is at least one mismatched base pair between the bound strand of the at least one dsNA molecule and the soluble strand of the at least one dsNA molecule in either or both of the recognition domain and the toehold domain.

6. The composition of any of items 1-5, wherein the quenchable colorimetric indicator is a fluorescent indicator.

7. The composition of any of items 1-6, wherein the soluble strand of the at least one dsNA molecule is configured to be displaced by a single stranded NA molecule, thereby displacing the quencher of the colorimetric indicator such that the colorimetric indicator is active.

8. The composition of item 7, wherein the active colorimetric indicator is detectable and/or quantifiable.

9. The composition of any of items 1-8, wherein the dsNA molecule comprises DNA, RNA, or LNA.

10. The composition of any of items 1-9, wherein the bound strand of the at least one dsNA molecule comprises a COVID-19-specific nucleic acid, or encodes a COVID-19-specific protein.

11. The composition of any of items 1-10, wherein the bound strand of the at least one dsNA molecule has a sequence selected from the group consisting of SEQ ID NOs: 1, 12, and 21; and
wherein the soluble strand of the at least one dsNA molecule has a sequence selected from the group consisting of SEQ ID NOs: 2-11, 13-20, and 22.

12. A kit for detecting the presence of a nucleic acid in a sample, the kit comprising:
microspheres; and
at least one double-stranded nucleic acid (dsNA) molecule,
wherein one strand of the at least one dsNA molecule is bound to an outer surface of the microspheres at a 3′end,
wherein the bound strand of the at least one dsNA molecule comprises a recognition domain and contains a quenchable colorimetric indicator at a 5′ end, and
wherein the other strand of the at least one dsNA molecule is soluble and contains a quencher of the colorimetric indicator at a 3′ end.

13. The kit of item 12, wherein the microspheres are coated in streptavidin and the bound strand of the at least one dsNA molecule comprises biotin at 3′ end.

14. The kit of items 12 or 13, wherein the bound strand of the at least one dsNA molecule further comprises a toehold domain at 3′ end.

15. The kit of item 14, wherein the toehold domain comprises between 1 and 12 nucleotides.

16. The kit of item 14 or 15, wherein there is at least one mismatched base pair between the bound strand of the at least one dsNA molecule and the soluble strand of the at least one dsNA molecule in either or both of the recognition domain and the toehold domain.

17. The kit of any of items 12-16, wherein the quenchable colorimetric indicator is a fluorescent indicator.

18. The kit of any of items 12-17, wherein the soluble strand of the at least one dsNA molecule is configured to be displaced by a single stranded NA molecule, thereby displacing the quencher of the colorimetric indicator such that the colorimetric indicator is active.

19. The kit of item 18, wherein the active colorimetric indicator is detectable and/or quantifiable.

20. The kit of any of items 12-19, wherein the dsNA molecule comprises DNA, RNA, or LNA.

21. The kit of any of items 12-20, wherein the bound strand of the at least one dsNA molecule comprises a COVID-19-specific nucleic acid, or encodes a COVID-19-specific protein.

22. The kit of any of items 12-21, wherein the bound strand of the at least one dsNA molecule has a sequence selected from the group consisting of SEQ ID NOs: 1, 12, and 21; and
wherein the soluble strand of the at least one dsNA molecule has a sequence selected from the group consisting of SEQ ID NOs: 2-11, 13-20, and 22.

23. The kit of any of items 12-22, wherein the sample comprises a biological sample, a water sample, a wastewater sample, an environmental sample, a food sample, a food contact surface sample, and/or a high surface contact area sample.

24. The kit of any of items 12-23, wherein the biological sample comprises tissue, blood, serum, plasma, saliva, nasopharyngeal swab or secretion, urine, cerebrospinal fluid, lymphatic fluid, sputum, cell lysate, and the like.

25. A method of detecting and/or quantifying a nucleic acid, the method comprising:
incubating the nucleic acid with a composition comprising:
microspheres; and
at least one double-stranded nucleic acid (dsNA) molecule,
wherein one strand of the at least one dsNA molecule is bound to an outer surface of the microspheres at a 3′end,
wherein the bound strand of the at least one dsNA molecule comprises a
recognition domain and contains a quenchable colorimetric indicator at a 5′ end,
wherein the other strand of the at least one dsNA molecule is soluble and contains a quencher of the colorimetric indicator at a 3′ end,
wherein the soluble strand of the at least one dsNA molecule is configured to be displaced by the nucleic acid, thereby displacing the quencher of the colorimetric indicator such that the colorimetric indicator is active; and
measuring the colorimetric indicator to detect and/or quantify the nucleic acid.

26. The method of item 25, wherein the measuring step is performed by a high-throughput spectrophotometer, a fluorescence activated cell sorting (FACS) system, flow cytometer, or a color activated cell sorting system.

27. The method of item 25 or 26, wherein the microspheres are coated in streptavidin and the bound strand of the at least one dsNA molecule comprises biotin at 3′ end.

28. The method of any of items 25-27, wherein the bound strand of the at least one dsNA molecule further comprises a toehold domain at 3′ end.

29. The method of item 28, wherein the toehold domain comprises between 1 and 12 nucleotides.

30. The method of item 28 or 29, wherein there is at least one mismatched base pair between the bound strand of the at least one dsNA molecule and the soluble strand of the at least one dsNA molecule in either or both of the recognition domain and the toehold domain.

31. The method of any of items 25-30, wherein the quenchable colorimetric indicator is a fluorescent indicator.

32. The method of any of items 25-31, wherein the dsNA molecule comprises DNA, RNA, or LNA.

33. The method of any of items 25-32, wherein the bound strand of the at least one dsNA molecule comprises a COVID-19-specific nucleic acid, or encodes a COVID-19-specific protein.

34. The method of any of items 25-33, wherein the bound strand of the at least one dsNA molecule has a sequence selected from the group consisting of SEQ ID NOs: 1, 12, and 21; and
wherein the soluble strand of the at least one dsNA molecule has a sequence selected from the group consisting of SEQ ID NOs: 2-11, 13-20, and 22.

35. A method of detecting a nucleic acid in a biological sample from a patient, the method comprising:
isolating the nucleic acid from the biological sample;
incubating the nucleic acid with a composition comprising:
   microspheres; and
at least one double-stranded nucleic acid (dsNA) molecule,
wherein one strand of the at least one dsNA molecule is bound to an outer
surface of the microspheres at a 3′end, wherein the bound strand of the at least one dsNA molecule comprises a
recognition domain and contains a quenchable colorimetric indicator at a 5′ end, wherein the other strand of the at least one dsNA molecule is soluble and
contains a quencher of the colorimetric indicator at a 3′ end, wherein the soluble strand of the at least one dsNA molecule is configured to
be displaced by the nucleic acid, thereby displacing the quencher of the colorimetric indicator such that the colorimetric indicator is active; and
measuring the colorimetric indicator to detect and/or quantify the nucleic acid.

36. The method of item 35, wherein the measuring step is performed by a high-throughput spectrophotometer, a fluorescence activated cell sorting (FACS) system, or a color activated cell sorting system.

37. The method of item 35 or 36, wherein the microspheres are coated in streptavidin and the bound strand of the at least one dsNA molecule comprises biotin at 3′ end.

38. The method of any of items 35-37, wherein the bound strand of the at least one dsNA molecule further comprises a toehold domain at 3′ end.

39. The method of item 38, wherein the toehold domain comprises between 1 and 12 nucleotides.

40. The method of item 38 or 39, wherein there is at least one mismatched base pair between the bound strand of the at least one dsNA molecule and the soluble strand of the at least one dsNA molecule in either or both of the recognition domain and the toehold domain.

41. The method of any of items 35-40, wherein the quenchable colorimetric indicator is a fluorescent indicator.

42. The method of any of items 35-41, wherein the dsNA molecule comprises DNA, RNA, or LNA.

43. The method of any of items 35-42, wherein the bound strand of the at least one dsNA molecule comprises a COVID-19-specific nucleic acid, or encodes a COVID-19-specific protein.

44. The method of any of items 35-43, wherein the bound strand of the at least one dsNA molecule has a sequence selected from the group consisting of SEQ ID NOs: 1, 12, and 21; and
wherein the soluble strand of the at least one dsNA molecule has a sequence selected from the group consisting of SEQ ID NOs: 2-11, 13-20, and 22.

45. The method of any of items 35-45, wherein the patient is a mammal.

46. The method of any of items 35-45, wherein the patient is a human or veterinary animal.

47. The method of any of items 35-46, wherein the biological sample comprises tissue, blood, serum, plasma, saliva, nasopharyngeal swab or secretion, urine, cerebrospinal fluid, lymphatic fluid, sputum, cell lysate, and the like.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A composition comprising:
a microsphere; and
a double-stranded nucleic acid (dsNA) molecule;
wherein one strand of the dsNA molecule is bound to an outer surface of the microsphere at a 3′ end;
wherein the bound strand comprises a recognition domain and contains a quenchable colorimetric indicator at a 5′ end; and
wherein the other strand of the dsNA molecule is soluble and contains a quencher of the colorimetric indicator at a 3′ end.

2. The composition of claim 1, wherein at least one of:
the microsphere is coated in streptavidin;
the bound strand further comprises biotin at 3′ end;
the bound strand further comprises a toehold domain at 3′ end;
the bound strand further comprises a COVID-19-specific nucleic acid, or encodes a COVID-19-specific protein;
the soluble strand is configured to be displaced by a single stranded NA molecule, thereby displacing the quencher of the colorimetric indicator such that the colorimetric indicator is active;
the quenchable colorimetric indicator is a fluorescent indicator; or
the dsNA molecule comprises DNA, RNA, or LNA.

3. (canceled)

4. The composition of claim 1, wherein the bound strand further comprises a toehold domain at 3′ end; and
wherein the toehold domain comprises between 1 and 15 nucleotides.

5. The composition of claim 4, wherein there is at least one mismatched base pair between the bound strand and the soluble strand in either or both of the recognition domain and the toehold domain.

6. (canceled)

7. The composition of claim 5, wherein the microsphere is coated in streptavidin;
wherein the bound strand further comprises biotin at 3′ end; and
wherein the soluble strand is configured to be displaced by a single stranded NA molecule, thereby displacing the quencher of the colorimetric indicator such that the colorimetric indicator is active.

8-10. (canceled)

11. A composition comprising:
microspheres; and
double-stranded nucleic acid (dsNA) molecules;
wherein at least a portion of the dsNA molecules have a bound strand and a soluble strand;
wherein each bound strand is bound to an outer surface of one of the microspheres at a 3′ end;
wherein each bound strand comprises a recognition domain, a toehold domain at 3′ end, and contains a quenchable colorimetric indicator at a 5′ end;
wherein each soluble strand is soluble and contains a quencher of the colorimetric indicator at a 3′ end;
wherein each bound strand has a sequence selected from the group consisting of SEQ ID NOs: 1, 12, and 21; and
wherein each soluble strand has a sequence selected from the group consisting of SEQ ID NOs: 2-11, 13-20, and 22.

12. A kit comprising:
the composition of claim 1.

13. A kit comprising:
the composition of claim 11.

14.-22. (canceled)

23. The kit of claim 12, wherein the kit is configured to detect a presence of a nucleic acid in a sample; and
wherein the sample comprises a biological sample, a water sample, a wastewater sample, an environmental sample, a food sample, a food contact surface sample, and/or a high surface contact area sample.

24. The kit of claim 23, wherein the biological sample comprises tissue, blood, serum, plasma, saliva, nasopharyngeal swab or secretion, urine, cerebrospinal fluid, lymphatic fluid, sputum, cell lysate, and the like.

25. A method of detecting and/or quantifying a nucleic acid comprising:
incubating the nucleic acid with the composition of claim 1;
wherein the soluble strand is configured to be displaced by the nucleic acid, thereby displacing the quencher of the colorimetric indicator such that the colorimetric indicator is active; and
measuring the colorimetric indicator to detect and/or quantify the nucleic acid.

26. The method of claim 25, wherein the measuring comprises measuring by a fluorescence activated cell sorting (FACS) system, flow cytometer, or a color activated cell sorting system.

27.-34. (canceled)

35. The method of claim 25 further comprising:
isolating the nucleic acid from a biological sample from a patient.

36. The method of claim 35, wherein the measuring comprises measuring by a fluorescence activated cell sorting (FACS) system, flow cytometer, or a color activated cell sorting system.

37.-47. (canceled)

48. A method of detecting and/or quantifying a nucleic acid comprising:
incubating the nucleic acid with the composition of claim 11;
wherein each soluble strand is configured to be displaced by the nucleic acid, thereby displacing the quencher of the colorimetric indicator such that the colorimetric indicator is active; and
measuring the colorimetric indicator to detect and/or quantify the nucleic acid.

49. The method of claim 48, wherein the measuring comprises measuring by a fluorescence activated cell sorting (FACS) system, flow cytometer, or a color activated cell sorting system.

50. A composition comprising:
probe-functionalized microspheres comprising:
microspheres; and
double-stranded (ds) DNA or locked nucleic acid (LNA) probes;
wherein the probes are immobilized on the microspheres; and
wherein the probes comprise a 6-carboxyfluorescein (FAM)-labeled immobilized sequence bound to a soluble, quencher-capped hybridization partner with a central abasic nucleotide.

51. A method comprising:
incubating the composition of claim 50 with unlabeled RNA; and
measuring fluorescence of the probe-functionalized microspheres using flow cytometry.

52. The method of claim 51, wherein the measured fluorescence of at least a portion of the probe-functionalized microspheres is a result of selective, toehold-mediated displacement of the quencher-capped hybridization partner by the unlabeled RNA.
